(19) United States
(12) Patent Application Publication
(10) Pub. No.: US 2024/0390250 A1
(43) Pub. Date: Nov. 28, 2024

(54) CLEANSING DYE COMPOSITIONS

(71) Applicant: Kao USA Inc., Cincinnati, OH (US)

(72) Inventor: Amy Davis, Cincinnati, OH (US)

(73) Assignee: Kao USA Inc., Cincinnati, OH (US)

(21) Appl. No.: 18/666,115

(22) Filed: May 16, 2024

Related U.S. Application Data
(60) Provisional application No. 63/504,310, filed on May 25, 2023.

(51) Int. Cl. A61K 8/60; A61K 8/44; A61K 8/46; A61Q 5/02; A61Q 5/06; A61Q 5/12

(52) U.S. Cl. CPC A61K 8/602; A61K 8/442; A61K 8/466; A61Q 5/02; A61Q 5/065; A61Q 5/12; A61K 2800/432

(57) ABSTRACT

Cleansing dye compositions are disclosed. In embodiments, a cleansing dye composition may include, based on a total weight of the cleansing dye composition, greater than or equal to 0.0001 wt % and less than or equal to 10 wt % of at least one direct dye; and greater than 15 wt % and less than or equal to 50 wt % of at least one cyclic saccharide-derived surfactant having a total surfactant activity level greater than 7.5 wt % and less than or equal to 25 wt %.

### CROSS-REFERENCE TO RELATED APPLICATIONS

The present specification claims the benefit of U.S. Provisional Application Ser. No. 63/504,310 filed May 25, 2023 and entitled “Cleansing Dye Compositions,” the entirety of which is incorporated by reference herein.

### FIELD

Embodiments of the present disclosure are generally related to dye compositions, and are specifically related to cleansing dye compositions having improved dye deposition.

### TECHNICAL BACKGROUND

Hair care product consumers may desire compositions that cleanse while also depositing color on the hair. However, conventional cleansing dye compositions may yield suboptimal color results and may also require high dye loads, resulting in staining, irritation, and poor environmental effects.

Accordingly, a need exists for alternative cleansing dye compositions that produce the desired dye deposition without requiring high dye loads.

### SUMMARY

According to a first aspect A1, a cleansing dye composition may comprise, based on a total weight of the cleansing dye composition, greater than or equal to 0.0001 wt % and less than or equal to 10 wt % of at least one direct dye; and greater than 15 wt % and less than or equal to 50 wt % of at least one cyclic saccharide-derived surfactant having a total surfactant activity level greater than 7.5 wt % and less than or equal to 25 wt %.

A second aspect A2 includes the cleansing dye composition of the first aspect A1, wherein the cleansing dye composition comprises, based on the total weight of the cleansing dye composition, greater than or equal to 17 wt % and less than or equal to 45 wt %, greater than or equal to 19 wt % and less than or equal to 40 wt %, or even greater than or equal to 21 wt % and less than or equal to 35 wt % of the at least one cyclic saccharide-derived surfactant.

A third aspect A3 includes the cleansing dye composition of the first aspect A1 or the second aspect A2, wherein the total surfactant activity level of the at least one cyclic saccharide-derived surfactant is, based on the total weight of the cleansing dye composition, greater than or equal to 8.5 wt % and less than or equal to 21 wt %, greater than or equal to 9.5 wt % and less than or equal to 19 wt %, or even greater than or equal to 10.5 wt % and less than or equal to 17 wt %.

A fourth aspect A4 includes the cleansing dye composition according to any one of the first through third aspects A1-A3, wherein the at least one cyclic saccharide-derived surfactant comprises decyl glucoside, coco glucoside, lauryl glucoside, sodium laurylglucoside hydroxypropylsulfonate, caprylyl/capryl glucoside, or combinations thereof.

A fifth aspect A5 includes the cleansing dye composition according to any one of the first through fourth aspects A1-A4, wherein the cleansing dye composition further comprises at least one secondary surfactant.

A sixth aspect A6 includes the cleansing dye composition according to the fifth aspect A5, wherein the secondary surfactant comprises, betaines, imidazoline derivatives, sarcosinates, sulfosuccinates, sulfates, sulfonates, amino acid-derived surfactants, isethionates, polysorbates, carboxylates, dimeric surfactants, alkoxylates, silicon surfactants, cationic surfactants, or combinations thereof.

A seventh aspect A7 includes the cleansing dye composition according to the fifth or the sixth aspects A5 or A6, wherein the cleansing dye composition comprises alkyl betaines, alkyl amido alkyl betaines, sulfo betaines, alkyl sulfates, alkyl ether sulfates, alkyl sulfonates, glutamates, fatty alcohol ethoxylates, alkyl phenol ethoxylates, fatty acid alkoxylates, alkyl ether carboxylates, silicon polyethers, quaternary ammonium compounds, amine salts, or combinations thereof.

An eighth aspect A8 includes the cleansing dye composition according to any one of the fifth through seventh aspects A5-A7, wherein the cleansing dye composition comprises cocamidopropyl betaine, sodium lauroyl sarcosinate, sodium cocoyl isethionate, sodium lauryl sulfoacetate, disodium laureth sulfosuccinate, sodium C14-C16 olefin sulfonate, sodium C16 olefin sulfonate, or combinations thereof.

A ninth aspect A9 includes the cleansing dye composition according to any one of the fifth through eighth aspects A5-A8, wherein the cleansing dye composition comprises a greater amount of the at least one cyclic saccharide-derived surfactant than the at least one secondary surfactant, based on the total weight of the cleansing dye composition.

A tenth aspect A10 includes the cleansing dye composition according to any one of the fifth through ninth aspects A5-A9, wherein the cleansing dye composition comprises a weight ratio of the at least one cyclic saccharide-derived surfactant to the at least one secondary surfactant of 1:1 to 5:1.

An eleventh aspect A11 includes the cleansing dye composition according to any one of the fifth through tenth aspects A5-A10, wherein the cleansing dye composition comprises, based on the total weight of the cleansing dye composition, greater than or equal to 1 wt % and less than or equal to 25 wt % of the secondary surfactant.

A twelfth aspect A12 includes the cleansing dye composition according to any one of the fifth through eleventh aspects A5-A11, wherein a total amount of the at least one cyclic saccharide-derived surfactant and the at least one secondary surfactant is, based on the total weight of the cleansing dye composition, greater than or equal to 16 wt % and less than or equal to 50 wt %.

A thirteenth aspect A13 includes the cleansing dye composition according to any one of the fifth through twelfth aspects A5-A12, wherein a total surfactant activity level of the at least one cyclic saccharide-derived surfactant and the at least one secondary surfactant that is, based on the total weight of the cleansing dye composition, greater than or equal to 8 wt % and less than or equal to 25 wt %.

A fourteenth aspect A14 includes the cleansing dye composition according to any one of the first through thirteenth aspects A1-A13, wherein the at least one direct dye comprises an acidic dye.

A fifteenth aspect A15 includes the cleansing dye composition according to any one of the first through fourteenth aspects A1-A14, wherein the at least one direct dye comprises Ext. Violet 2/Acid Violet 43, Blue 1/Acid Blue 9, FD&C Green 3, D&C Green 5, D&C Green 6, D&C Green 8, D&C Yellow 8, Yellow 11, Yellow 10/Acid Yellow 3, Yellow 5/Acid Yellow 23, FD&C Yellow 6/E110, Orange 4/Acid Orange 7, Orange 5, Red 6 FD&C Red 40, D&C Red 4, D&C Red 6, D&C Red 17, D&C Red 33/Acid Red 33, D&C Red 27, D&C Red 28, Violet 2, HC Blue 2, 2-amino-6-chloro-4-nitrophenol, HC Red No. 3, HC Blue No. 12, 4-hydroxypropylamino-3-nitrophenol, Basic Yellow 57, Basic Red 76, Basic Blue 124, Basic Violet 2, Basic Blue 99, HC Blue 16, Basic Orange 31, Basic Brown 17, Basic Red 51, Basic Yellow 87 or combinations thereof.

A sixteenth aspect A16 includes the cleansing dye composition according to any one of the first through fifteenth aspects A1-A15, wherein the cleansing dye composition comprises, based on the total weight of the cleansing dye composition, greater than or equal to 0.001 wt % and less than or equal to 7 wt %, greater than or equal to 0.005 wt % and less than or equal to 5 wt %, greater than or equal to 0.01 wt % and less than or equal to 3 wt %, or greater than or equal to 0.05 wt % and less than or equal to 1 wt % of the at least one direct dye.

A seventeenth aspect A17 includes the cleansing dye composition according to any one of the first through sixteenth aspects A1-A16, wherein the cleansing dye composition further comprises, based on the total weight of the cleansing dye composition, greater than or equal to 0.05 wt % and less than or equal to 2 wt % of at least one pH adjuster.

An eighteenth aspect A18 includes the cleansing dye composition according to any one of the first through seventeenth aspects A1-A17, wherein the cleansing dye composition further comprises, based on the total weight of the cleansing dye composition, greater than or equal to 0.05 wt % and less than or equal to 5 wt % of at least one conditioning agent.

A nineteenth aspect A19 includes the cleansing dye composition according to any one of the first through eighteenth aspects A1-A18, wherein the cleansing dye composition further comprises, based on the total weight of the cleansing dye composition, greater than or equal to 0.005 wt % and less than or equal to 5 wt % of at least one foaming agent.

A twentieth aspect A20 includes the cleansing dye composition according to any one of the first through nineteenth aspects A1-A19, wherein the cleansing dye composition further comprises, based on the total weight of the cleansing dye composition, greater than or equal to 0.1 wt % and less than or equal to 3 wt % of at least one humectant.

A twenty first aspect A21 includes the cleansing dye composition according to any one of the first through twentieth aspects A1-A20, wherein the cleansing dye composition is formulated for use as a shampoo.

A twenty second aspect A22 includes a method for coloring a keratin substrate, comprising applying to the keratin substrate the cleansing dye composition according to any one of the first through twenty first aspects A1-A21.

Additional features and advantages of the cleansing dye compositions described herein will be set forth in the detailed description which follows, and in part will be readily apparent to those skilled in the art from that description or recognized by practicing the embodiments described herein, including the detailed description which follows and the claims.

It is to be understood that both the foregoing general description and the following detailed description describe various embodiments and are intended to provide an overview or framework for understanding the nature and character of the claimed subject matter.

### DETAILED DESCRIPTION

Reference will now be made in detail to various embodiments of cleansing dye compositions that have improved dye deposition, as evidenced by increased color change (e.g., ΔE and CIELAB color space values). According to embodiments, a cleansing dye composition includes greater than or equal to 0.0001 wt % and less than or equal to 10 wt % of at least one direct dye; and greater than 15 wt % and less than or equal to 50 wt % of at least one cyclic saccharide-derived surfactant having a total surfactant activity level greater than 7.5 wt % and less than or equal to 25 wt %. Various embodiments of cleansing dye compositions will be described herein.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, or it is not otherwise specifically stated in the claims or description that the steps are to be limited to a specific order, or that a specific order or orientation to components of an apparatus is not recited, it is in no way intended that an order or orientation be inferred, in any respect. This holds for any possible non-express basis for interpretation, including: matters of logic with respect to arrangement of steps, operational flow, order of components, or orientation of components; plain meaning derived from grammatical organization or punctuation, and; the number or type of embodiments described in the specification.

As used herein, the singular forms “a,” “an” and “the” include plural referents unless the context clearly dictates otherwise. Thus, for example, reference to “a” component includes aspects having two or more such components, unless the context clearly indicates otherwise.

In the embodiments of the cleansing dye compositions described herein, the amounts of components (e.g., direct dye, cyclic saccharide-derived surfactant, and the like) are specified in weight percent (wt %), unless otherwise specified.

The terms “0 wt %” and “free,” when used to describe the amount and/or absence of a particular component in a cleansing dye composition, means that the component is not intentionally added to the cleansing dye composition. However, the cleansing dye composition may contain traces of the component as a contaminant or tramp in amounts less than 0.05 wt %.

The term “ΔE value,” as described herein, refers to a measure of change in visual perception of two given colors, as calculated according to the equation, ΔE*_{ab}=\sqrt{(L*₂−L*₁)²+(a*₂−a*₁)²+(b*₂−b*₁)²}, where CIELAB color space values were measured according to standard method using a Konica Minolta CR-400 Chromameter with D65 illumination and an observer angle of 2°. A relatively greater ΔE value corresponds to a greater change in the color of a tress upon treatment with a cleansing dye composition.

The term “CIELAB color space,” as used herein, refers to a color space defined by the International Commission on Illumination (CIE) in 1976. The CIELAB color space expresses color as three values: L* for the lightness from black (0) to white (100), a* from green (−) to red (+), and b* from blue (−) to yellow (+). Accordingly, a tress with a lower b* value correlates to more blue direct dye being deposited onto the tress.

The term “active,” as used herein, refers to the active amount of the surfactant, based on a total amount of the raw material.

The terms “total activity level” or “total surfactant activity level,” as used herein, refer to the activity level of the surfactant, in terms of wt %, in the cleansing dye composition, based on a total weight of the cleansing dye composition.

Non-reactive direct dye-depositing formulas are frequently used in mass market consumer products for dyeing a keratin substrate, such as hair or skin. The quality of dye deposition is dependent on the solubility of the dye in the coloring formula. Dyes that are not solubilized enough result in streaky, uneven color deposition; dyes that are overly-solubilized have a greater affinity for the formula than the substrate, and, consequently, will not deposit onto the substrate.

The surfactants used in conventional dye cleansing products to solubilize the dyes in the base composition of a coloring formula may be present in a relatively high amount such that the dyes have a diminished affinity for the substrate. This may lead to poor color transformation. As a result, conventional color-enhancing cleansing products may require high dye loads and long leave-on times to maximize color deposition. This increases the likelihood of staining and irritation and also causes such products to have poor environmental effects (e.g., lower biodegradability and higher ccotoxicity profiles). In an attempt to overcome these issues, penetration enhancers, like propylene glycol, may be added to such products. Yet, even under these conditions, conventional formulas may only achieve sub-optimal results. Accordingly, there is a need for dye cleansing products that maximize dye deposition while enhancing consumer experience and reducing environmental impact.

Disclosed herein are cleansing dye compositions that mitigate the aforementioned problems such that the desired amount of dye deposition may be achieved without requiring higher dye loads. Specifically, the cleansing dye compositions disclosed herein include at least one direct dye and at least one cyclic saccharide-derived surfactant. The at least one cyclic saccharide-derived surfactant improves dye deposition, as evidenced by increased color change (i.e., ΔE and CIELAB color space values). A minimum amount of the at least one cyclic saccharide-derived surfactant (e.g., greater than 15 wt %) is included in the cleansing dye composition to ensure that the composition is sufficiently solubilized and the desired color is achieved. The amount of the at least one cyclic saccharide-derived surfactant in the cleansing dye composition may be minimized (e.g., less than or equal to 50 wt %) to ensure that the composition is not overly-solubilized. The at least one cyclic saccharide-derived surfactants used herein have a relatively high total surfactant activity level (e.g., greater than 7.5 wt %) such that a relatively low amount of the cyclic saccharide-derived surfactant may be used as compared to the amount of surfactant in conventional dye cleansing compositions.

The cleansing dye compositions disclosed herein may generally be described as comprising direct dye and cyclic saccharide-derived surfactant.

### Direct Dye

The cleansing dye compositions described herein include dye to impart color.

In embodiments, the at least one direct dye may comprise an acidic dye, a basic dye, a nitro dye, or combinations thereof. In embodiments, the at least one direct dye may comprise Ext. Violet 2/Acid Violet 43, Blue 1/Acid Blue 9, FD&C Green 3, D&C Green 5, D&C Green 6, D&C Green 8, D&C Yellow 8, Yellow 11, Yellow 10/Acid Yellow 3, Yellow 5/Acid Yellow 23, FD&C Yellow 6/E110, Orange 4/Acid Orange 7, Orange 5, Red 6 FD&C Red 40, D&C Red 4, D&C Red 6, D&C Red 17, D&C Red 33/Acid Red 33, D&C Red 27, D&C Red 28, Violet 2, HC Blue 2, 2-amino-6-chloro-4-nitrophenol, HC Red No. 3, HC Blue No. 12, 4-hydroxypropylamino-3-nitrophenol, Basic Yellow 57, Basic Red 76, Basic Blue 124, Basic Violet 2, Basic Blue 99, HC Blue 16, Basic Orange 31, Basic Brown 17, Basic Red 51, Basic Yellow 87, or combinations thereof.

In embodiments, the cleansing dye composition may comprise, based on a total weight of the cleansing dye composition, greater than or equal to 0.0001 wt % and less than or equal to 10 wt % of at least one direct dye. In embodiments, the amount of the at least one direct dye in the cleansing dye composition may be, based on a total weight of the cleansing dye composition, greater than or equal to 0.0001 wt %, greater than or equal to 0.001 wt %, greater than or equal to 0.01 wt %, or even greater than or equal to 0.05 wt %. In embodiments, the amount of the at least one direct dye in the cleansing dye composition may be, based on a total weight of the cleansing dye composition, less than or equal to 10 wt %, less than or equal to 7 wt %, less than or equal to 5 wt %, less than or equal to 3 wt %, or even less than or equal to 1 wt %. In embodiments, the amount of the at least one direct dye in the cleansing dye composition may be, based on a total weight of the cleansing dye composition, greater than or equal to 0.0001 wt % and less than or equal to 10 wt %, greater than or equal to 0.0001 wt % and less than or equal to 7 wt %, greater than or equal to 0.0001 wt % and less than or equal to 5 wt %, greater than or equal to 0.0001 wt % and less than or equal to 3 wt %, greater than or equal to 0.0001 wt % and less than or equal to 1 wt %, greater than or equal to 0.001 wt % and less than or equal to 10 wt %, greater than or equal to 0.001 wt % and less than or equal to 7 wt %, greater than or equal to 0.001 wt % and less than or equal to 5 wt %, greater than or equal to 0.001 wt % and less than or equal to 3 wt %, greater than or equal to 0.001 wt % and less than or equal to 1 wt %, greater than or equal to 0.01 wt % and less than or equal to 10 wt %, greater than or equal to 0.01 wt % and less than or equal to 7 wt %, greater than or equal to 0.01 wt % and less than or equal to 5 wt %, greater than or equal to 0.01 wt % and less than or equal to 3 wt %, greater than or equal to 0.01 wt % and less than or equal to 1 wt %, greater than or equal to 0.05 wt % and less than or equal to 10 wt %, greater than or equal to 0.05 wt % and less than or equal to 7 wt %, greater than or equal to 0.05 wt % and less than or equal to 5 wt %, greater than or equal to 0.05 wt % and less than or equal to 3 wt %, or even greater than or equal to 0.05 wt % and less than or equal to 1 wt %, or any and all sub-ranges formed from any of these endpoints.

### Cyclic Saccharide-Derived Surfactant

The at least one cyclic saccharide-derived surfactant included in the cleansing dye composition described herein improves dye deposition, as evidenced by increased color change (e.g., ΔE and CIELAB color space values). While not wishing to be bound by theory, it is believed that the structure of the cyclic saccharide-derived surfactant results in the improved dye deposition.

In embodiments, the at least one cyclic saccharide-derived surfactant may comprise decyl glucoside, coco glucoside, lauryl glucoside, sodium laurylglucoside hydroxypropylsulfonate, caprylyl/capryl glucoside, or combinations thereof.

As described hereinabove, the quality of dye deposition is dependent on the solubility of the dye in the coloring formula. Dyes that are not solubilized enough result in streaky, uneven color deposition; dyes that are overly-solubilized have a greater affinity for the formula than the substrate, and, consequently, will not deposit onto the substrate. Accordingly, a minimum amount of the at least one cyclic saccharide-derived surfactant (e.g., greater than 15 wt %) is included in the cleansing dye composition to ensure that the composition is sufficiently solubilized and the desired color is achieved. The amount of the at least one cyclic saccharide-derived surfactant in the cleansing dye composition may be minimized (e.g., less than or equal to 50 wt %) to ensure that the composition is not overly-solubilized.

In embodiments, the cleansing dye composition may comprise, based on a total weight of the cleansing dye composition, greater than 15 wt % and less than or equal to 50 wt % of the at least one cyclic saccharide-derived surfactant. In embodiments, the amount of the at least one cyclic saccharide-derived surfactant in the cleansing dye composition may be, based on a total weight of the cleansing dye composition, greater than 15 wt %, greater than or equal to 17 wt %, greater than or equal to 19 wt %, or even greater than or equal to 21 wt %. In embodiments, the amount of the at least one cyclic saccharide-derived surfactant in the cleansing dye composition may be, based on a total weight of the cleansing dye composition, less than or equal to 50 wt %, less than or equal to 45 wt %, less than or equal to 40 wt %, or even less than or equal to 35 wt %. In embodiments, the amount of the at least cyclic saccharide-derived surfactant in the cleansing dye composition may be, based on a total weight of the cleansing dye composition, greater than 15 wt % and less than or equal to 50 wt %, greater than 15 wt % and less than or equal to 45 wt %, greater than 15 wt % and less than or equal to 40 wt %, greater than 15 wt % and less than or equal to 35 wt %, greater than or equal to 17 wt % and less than or equal to 50 wt %, greater than or equal to 17 wt % and less than or equal to 45 wt %, greater than or equal to 17 wt % and less than or equal to 40 wt %, greater than or equal to 17 wt % and less than or equal to 35 wt %, greater than or equal to 19 wt % and less than or equal to 50 wt %, greater than or equal to 19 wt % and less than or equal to 45 wt %, greater than or equal to 19 wt % and less than or equal to 40 wt %, greater than or equal to 19 wt % and less than or equal to 35 wt %, greater than or equal to 21 wt % and less than or equal to 50 wt %, greater than or equal to 21 wt % and less than or equal to 45 wt %, greater than or equal to 21 wt % and less than or equal to 40 wt %, or even greater than or equal to 21 wt % and less than or equal to 35 wt %, or any and all sub-ranges formed from any of these endpoints.

The at least one cyclic saccharide-derived surfactants used herein have a relatively high total surfactant activity level (e.g., greater than 7.5 wt %) such that a relatively low amount of the cyclic saccharide-derived surfactant may be used as compared to the amount of surfactant in conventional dye cleansing compositions. In embodiments, the cleansing dye composition may comprise, based on a total weight of the cleansing dye composition, a total surfactant activity level of the at least one cyclic saccharide-derived surfactant greater than 7.5 wt % and less than or equal to 25 wt %. In embodiments, the cleansing dye composition may comprise, based on a total weight of the cleansing dye composition, a total surfactant activity level of the at least one cyclic saccharide-derived surfactant greater than 7.5 wt %, greater than or equal to 8.5 wt %, greater than or equal to 9.5 wt %, or even greater than or equal to 10.5 wt %. In embodiments, the cleansing dye composition may comprise, based on a total weight of the cleansing dye composition, a total surfactant activity level of the at least one cyclic saccharide-derived surfactant less than or equal to 25 wt %, less than or equal to 21 wt %, less than or equal to 19 wt %, or even less than or equal to 17%. In embodiments, the total surfactant activity level of the at least one cyclic saccharide-derived surfactant may be, based on a total weight of the cleansing dye composition, greater than 7.5 wt % and less than or equal to 25 wt %, greater than 7.5 wt % and less than or equal to 21 wt %, greater than 7.5 wt % and less than or equal to 19 wt %, greater than 7.5 wt % and less than or equal to 17 wt %, greater than or equal to 8.5 wt % and less than or equal to 25 wt %, greater than or equal to 8.5 wt % and less than or equal to 21 wt %, greater than or equal to 8.5 wt % and less than or equal to 19 wt %, greater than or equal to 8.5 wt % and less than or equal to 17 wt %, greater than or equal to 9.5 wt % and less than or equal to 25 wt %, greater than or equal to 9.5 wt % and less than or equal to 21 wt %, greater than or equal to 9.5 wt % and less than or equal to 19 wt %, greater than or equal to 9.5 wt % and less than or equal to 17 wt %, greater than or equal to 10.5 wt % and less than or equal to 25 wt %, greater than or equal to 10.5 wt % and less than or equal to 21 wt %, greater than or equal to 10.5 wt % and less than or equal to 19 wt %, or even greater than or equal to 10.5 wt % and less than or equal to 17 wt %, or any and all sub-ranges formed from any of these endpoints.

### Secondary Surfactant

The addition of a secondary surfactant to a cleansing dye composition can further enhance the color results while imparting certain cleansing properties and foam aesthetics to the composition. In embodiments, the cleansing dye composition may comprise at least one secondary surfactant. In such embodiments, the secondary surfactant may comprise betaines, imidazoline derivatives, sarcosinates, sulfosuccinates, sulfates, sulfonates, amino acid-derived surfactants, isethionates, polysorbates, carboxylates, dimeric surfactants, alkoxylates, silicon surfactants, cationic surfactants, or combinations thereof. In some embodiments, the secondary surfactant may comprise alkyl betaines, alkyl amido alkyl betaines, sulfo betaines, alkyl sulfates, alkyl ether sulfates, alkyl sulfonates, glutamates, fatty alcohol ethoxylates, alkyl phenol ethoxylates, fatty acid alkoxylates, alkyl ether carboxylates, silicon polyethers, quaternary ammonium compounds, amine salts, or combinations thereof. In some embodiments, the secondary surfactant may comprise cocamidopropyl betaine, sodium lauroyl sarcosinate, sodium cocoyl isethionate, sodium lauryl sulfoacetate, disodium laureth sulfosuccinate, sodium C₁₄-C₁₆ olefin sulfonate, sodium C₁₆ olefin sulfonate, or combinations thereof.

In embodiments, the cleansing dye composition may comprise, based on a total weight of the cleansing dye composition, a greater amount of the at least one cyclic saccharide-derived surfactant than the at least one secondary surfactant. In embodiments, the weight ratio of the at least one cyclic saccharide-derived surfactant to the at least one secondary surfactant may be, based on a total weight of the cleansing dye composition, 1:1 to 5:1. In embodiments, the weight ratio of the at least one cyclic saccharide-derived surfactant to the at least one secondary surfactant may be, based on a total weight of the cleansing dye composition, greater than or equal to 1:1, greater than or equal to 2:1, greater than or equal to 3:1, or even greater than or equal to 4:1. In embodiments, the weight ratio of the at least one cyclic saccharide-derived surfactant to the at least one secondary surfactant may be, based on a total weight of the cleansing dye composition, less than or equal to 5:1, less than or equal to 4:1, less than or equal to 3:1, or even less than or equal to 2:1. In embodiments, the weight ratio of the at least one cyclic saccharide-derived surfactant to the at least one secondary surfactant may be, based on a total weight of the cleansing dye composition, from 1:1 to 5:1, from 1:1 to 4:1, from 1:1 to 3:1, form 1:1 to 2:1, from 2:1 to 5:1, from 2:1 to 4:1, from 2:1 to 3:1, from 3:1 to 5:1, from 3:1 to 4:1, or even from 4:1 to 5:1, or any and all sub-ranges formed from any of these endpoints.

In embodiments, the cleansing dye composition may comprise, based on a total weight of the cleansing dye composition, greater than or equal to 1 wt % and less than or equal to 25 wt % of the at least one secondary surfactant. In embodiments, the amount of the at least one secondary surfactant in the cleansing dye composition may be, based on a total weight of the cleansing dye composition, greater than or equal to 1 wt %, greater than or equal to 5 wt %, greater than or equal to 10 wt %, greater than or equal to 15 wt %, or even greater than or equal to 20 wt %. %. In embodiments, the amount of the at least one secondary surfactant in the cleansing dye composition may be, based on a total weight of the cleansing dye composition, less than or equal to 25 wt %, less than or equal to 20 wt %, less than or equal to 15 wt %, less than or equal to 10 wt %, or less than or equal to 5 wt %. In embodiments, the amount of the at least one secondary surfactant in the cleansing dye composition may be, based on a total weight of the cleansing dye composition, greater than or equal to 1 wt % and less than or equal to 25 wt %, greater than or equal to 1 wt % and less than or equal to 20 wt %, greater than or equal to 1 wt % and less than or equal to 15 wt %, greater than or equal to 1 wt % and less than or equal to 10 wt %, greater than or equal to 1 wt % and less than or equal to 5 wt %, greater than or equal to 5 wt % and less than or equal to 25 wt %, greater than or equal to 5 wt % and less than or equal to 20 wt %, greater than or equal to 5 wt % and less than or equal to 15 wt %, greater than or equal to 5 wt % and less than or equal to 10 wt %, greater than or equal to 10 wt % and less than or equal to 25 wt %, greater than or equal to 10 wt % and less than or equal to 20 wt %, greater than or equal to 10 wt % and less than or equal to 15 wt %, greater than or equal to 15 wt % and less than or equal to 25 wt %, greater than or equal to 15 wt % and less than or equal to 20 wt %, or even greater than or equal to 20 wt % and less than or equal to 25 wt %, or any and all sub-ranges formed from any of these endpoints.

In embodiments, the cleansing dye composition may comprise, based on the weight of the cleansing dye composition, a total amount of the at least one cyclic saccharide-derived surfactant and the at least one secondary surfactant greater than or equal to 16 wt % and less than or equal to about 50 wt %. In embodiments, the total amount of the at least one cyclic saccharide-derived surfactant and the at least one secondary surfactant in the cleansing dye composition may be, based on a total weight of the cleansing dye composition, greater than or equal to 16 wt %, greater than or equal to 20 wt %, greater than or equal to 25 wt %, greater than or equal to 30 wt %, greater than or equal to 35 wt %, greater than or equal to 40 wt %, or even greater than or equal to 45 wt %. In embodiments, the total amount of the at least one cyclic saccharide-derived surfactant and the at least one secondary surfactant in the cleansing dye composition may be, based on a total weight of the cleansing dye composition, less than or equal to 50 wt %, less than or equal to 45 wt %, less than or equal to 40 wt %, less than or equal to 35 wt %, less than or equal to 30 wt %, less than or equal to 25 wt %, or even less than or equal to 20%. In embodiments, the total amount of the at least one cyclic saccharide-derived surfactant and the at least one secondary surfactant in the cleansing dye composition may be, based on a total weight of the cleansing dye composition, greater than or equal to 16 wt % and less than or equal to 50 wt %, greater than or equal to 16 wt % and less than or equal to 45 wt %, greater than or equal to 16 wt % and less than or equal to 40 wt %, greater than or equal to 16 wt % and less than or equal to 35 wt %, greater than or equal to 16 wt % and less than or equal to 30 wt %, greater than or equal to 16 wt % and less than or equal to 25 wt %, greater than or equal to 16 wt % and less than or equal to 20 wt %, greater than or equal to 20 wt % and less than or equal to 50 wt %, greater than or equal to 20 wt % and less than or equal to 45 wt %, greater than or equal to 20 wt % and less than or equal to 40 wt %, greater than or equal to 20 wt % and less than or equal to 35 wt %, greater than or equal to 20 wt % and less than or equal to 30 wt %, greater than or equal to 20 wt % and less than or equal to 25 wt %, greater than or equal to 25 wt % and less than or equal to 50 wt %, greater than or equal to 25 wt % and less than or equal to 45 wt %, greater than or equal to 25 wt % and less than or equal to 40 wt %, greater than or equal to 25 wt % and less than or equal to 35 wt %, greater than or equal to 25 wt % and less than or equal to 30 wt %, greater than or equal to 30 wt % and less than or equal to 50 wt %, greater than or equal to 30 wt % and less than or equal to 45 wt %, greater than or equal to 30 wt % and less than or equal to 40 wt %, greater than or equal to 30 wt % and less than or equal to 35 wt %, greater than or equal to 35 wt % and less than or equal to 50 wt %, greater than or equal to 35 wt % and less than or equal to 45 wt %, greater than or equal to 35 wt % and less than or equal to 40 wt %, greater than or equal to 40 wt % and less than or equal to 50 wt %, greater than or equal to 40 wt % and less than or equal to 45 wt %, or even greater than or equal to 45 wt % and less than or equal to 50 wt %, or any and all sub-ranges formed from any of these endpoints.

In embodiments, the cleansing dye composition may comprise, based on the weight of the cleansing dye composition, a total surfactant activity level of the at least one cyclic saccharide-derived surfactant and the at least one secondary surfactant greater than or equal to 8 wt % and less than or equal to 25 wt %. In embodiments, the cleansing dye composition may comprise, based on the weight of the cleansing dye composition, a total surfactant activity level of the at least one cyclic saccharide-derived surfactant and the at least one secondary surfactant greater than or equal to 8 wt %, greater than or equal to 10 wt %, greater than or equal to 15 wt %, or even greater than or equal to 20 wt %. In embodiments, the cleansing dye composition may comprise, based on the weight of the cleansing dye composition, a total surfactant activity level of the at least one cyclic saccharide-derived surfactant and the at least one secondary surfactant less than or equal to 25 wt %, less than or equal to 20 wt %, less than or equal to 15 wt %, or even less than or equal to 10 wt %. In embodiments, the cleansing dye composition may comprise, based on the weight of the cleansing dye composition, a total surfactant activity level of the at least one cyclic saccharide-derived surfactant and the at least one secondary surfactant greater than or equal to 8 wt % and less than or equal to 25%, greater than or equal to 8 wt % and less than or equal to 20 wt %, greater than or equal to 8 wt % and less than or equal to 15 wt %, greater than or equal to 8 wt % and less than or equal to 10 wt %, greater than or equal to 10 wt % and less than or equal to 25 wt %, greater than or equal to 10 wt % and less than or equal to 20 wt %, greater than or equal to 10 wt % and less than or equal to 15 wt %, greater than or equal to 15 wt % and less than or equal to 25 wt %, greater than or equal to 15 wt % and less than or equal to 20 wt %, or even greater than or equal to 20 wt % and less than or equal to 25 wt %, or any and all sub-ranges formed from any of these endpoints.

### Additives

In embodiments, the cleansing dye composition may further comprise at least one additive, such as pH adjusters, conditioning agents, foaming agents, humectants, and combinations thereof.

In embodiments, the pH adjuster may comprise malic acid, citric acid, lactic acid, or combinations thereof. In embodiments, the amount of the pH adjuster in the cleansing dye composition may be, based on a total weight of the cleansing dye composition, greater than or equal to 0.05 wt % and less than or equal to 2 wt %. In embodiments, the amount of the pH adjuster in the cleansing dye composition may be, based on a total weight of the cleansing dye composition, greater than or equal to 0.05 wt %, greater than or equal to 0.1 wt %, greater than or equal to 0.5 wt %, greater than or equal to 1 wt %, or even greater than or equal to 1.5 wt %. In embodiments, the amount of the pH adjuster in the cleansing dye composition may be, based on a total weight of the cleansing dye composition, less than or equal to 2 wt %, less than or equal to 1.5 wt %, less than or equal to 1 wt %, less than or equal to 0.5 wt %, or even less than or equal to 0.1 wt %. In embodiments, the amount of the pH adjuster in the cleansing dye composition may be, based on a total weight of the cleansing dye composition, greater than or equal to 0.05 wt % and less than or equal to 2 wt %, greater than or equal to 0.05 wt % and less than or equal to 1.5 wt %, greater than or equal to 0.05 wt % and less than or equal to 1 wt %, greater than or equal to 0.05 wt % and less than or equal to 0.5 wt %, greater than or equal to 0.05 wt % and less than or equal to 0.1 wt %, greater than or equal to 0.1 wt % and less than or equal to 2 wt %, greater than or equal to 0.1 wt % and less than or equal to 1.5 wt %, greater than or equal to 0.1 wt % and less than or equal to 1 wt %, greater than or equal to 0.1 wt % and less than or equal to 0.5 wt %, greater than or equal to 0.5 wt % and less than or equal to 2 wt %, greater than or equal to 0.5 wt % and less than or equal to 1.5 wt %, greater than or equal to 0.5 wt % and less than or equal to 1 wt %, greater than or equal to 1 wt % and less than or equal to 2 wt %, greater than or equal to 1 wt % and less than or equal to 1.5 wt %, or even greater than or equal to 1.5 wt % and less than or equal to 2 wt %, or any and all sub-ranges formed from any of these endpoints.

In embodiments, the conditioning agent may comprise polyquaternium-7, polyquaternium-39, polyquaternium-10, hydroxypropyl methylcellulose, guar hydroxypropyl-trimonium chloride, or combinations thereof. In embodiments, the amount of the at least one conditioning agent in the cleansing dye composition may be, based on a total weight of the cleansing dye composition, greater than or equal to 0.05 wt % and less than or equal to 5 wt %. In embodiments, the amount of the at least one conditioning agent in the cleansing dye composition may be, based on a total weight of the cleansing dye composition, greater than or equal to 0.05 wt %, greater than or equal to 0.5 wt %, greater than or equal to 1 wt %, greater than or equal to 2 wt %, greater than or equal to 3 wt %, or even greater than or equal to 4 wt %. In embodiments, the amount of the at least one conditioning agent in the cleansing dye composition may be, based on a total weight of the cleansing dye composition, less than or equal to 5 wt %, less than or equal to 4 wt %, less than or equal to 3 wt %, less than or equal to 2 wt %, less than or equal to 1 wt %, or even less than or equal to 0.5 wt %. In embodiments, the amount of the at least one conditioning agent in the cleansing dye composition may be, based on a total weight of the cleansing dye composition, greater than or equal to 0.05 wt % and less than or equal to 5 wt %, greater than or equal to 0.05 wt % and less than or equal to 4 wt %, greater than or equal to 0.05 wt % and less than or equal to 3 wt %, greater than or equal to 0.05 wt % and less than or equal to 2 wt %, greater than or equal to 0.05 wt % and less than or equal to 1 wt %, greater than or equal to 0.5 wt % and less than or equal to 5 wt %, greater than or equal to 0.5 wt % and less than or equal to 4 wt %, greater than or equal to 0.5 wt % and less than or equal to 3 wt %, greater than or equal to 0.5 wt % and less than or equal to 2 wt %, greater than or equal to 0.5 wt % and less than or equal to 1 wt %, greater than or equal to 1 wt % and less than or equal to 5 wt %, greater than or equal to 1 wt % and less than or equal to 4 wt %, greater than or equal to 1 wt % and less than or equal to 3 wt %, greater than or equal to 1 wt % and less than or equal to 2 wt %, greater than or equal to 2 wt % and less than or equal to 5 wt %, greater than or equal to 2 wt % and less than or equal to 4 wt %, greater than or equal to 2 wt % and less than or equal to 3 wt %, greater than or equal to 3 wt % and less than or equal to 5 wt %, greater than or equal to 3 wt % and less than or equal to 4 wt %, or even greater than or equal to 4 wt % and less than or equal to 5 wt %, or any and all sub-ranges formed from any of these endpoints.

In embodiments, the foaming agent may comprise PEG-12 dimethicone, polyester-11, PPG-9, water (and) silicone quaternium-18 (and) trideceth-6 (and) trideceth-12, cocamide MEA, cetyl alcohol, glycol distearate, or combinations thereof. In embodiments, the amount of the at least one foaming agent in the cleansing dye composition may be, based on a total weight of the cleansing dye composition, greater than or equal to 0.005 wt % and less than or equal to 5 wt %. In embodiments, the amount of the at least one foaming agent may be, based on a total weight of the cleansing dye composition, greater than or equal to 0.005 wt %, greater than or equal to 0.5 wt %, greater than or equal to 1 wt %, greater than or equal to 2 wt %, greater than or equal to 3 wt %, or even greater than or equal to 4 wt %. In embodiments, the amount of the at least one foaming agent in the cleansing dye composition may be, based on a total weight of the cleansing dye composition, less than or equal to 5 wt %, less than or equal to 4 wt %, less than or equal to 3 wt %, less than or equal to 2 wt %, less than or equal to 1 wt %, or even less than or equal to 0.5 wt %. In embodiments, the amount of the at least one foaming agent in the cleansing dye composition may be, based on a total weight of the cleansing dye composition, greater than or equal to 0.005 wt % and less than or equal to 5 wt %, greater than or equal to 0.005 wt % and less than or equal to 4 wt %, greater than or equal to 0.005 wt % and less than or equal to 3 wt %, greater than or equal to 0.005 wt % and less than or equal to 2 wt %, greater than or equal to 0.005 wt % and less than or equal to 1 wt %, greater than or equal to 0.005 wt % and less than or equal to 0.5 wt %, greater than or equal to 0.5 wt % and less than or equal to 5 wt %, greater than or equal to 0.5 wt % and less than or equal to 4 wt %, greater than or equal to 0.5 wt % and less than or equal to 3 wt %, greater than or equal to 0.5 wt % and less than or equal to 2 wt %, greater than or equal to 0.5 wt % and less than or equal to 1 wt %, greater than or equal to 1 wt % and less than or equal to 5 wt %, greater than or equal to 1 wt % and less than or equal to 4 wt %, greater than or equal to 1 wt % and less than or equal to 3 wt %, greater than or equal to 1 wt % and less than or equal to 2 wt %, greater than or equal to 2 wt % and less than or equal to 5 wt %, greater than or equal to 2 wt % and less than or equal to 4 wt %, greater than or equal to 2 wt % and less than or equal to 3 wt %, greater than or equal to 3 wt % and less than or equal to 5 wt %, greater than or equal to 3 wt % and less than or equal to 4 wt %, or even greater than or equal to 4 wt % and less than or equal to 5 wt %, or any and all sub-ranges formed from any of these endpoints.

In embodiments, the humectant may comprise glycerin, propanediol, propylene glycol, butylene glycol, pentylene glycol, benzyl alcohol, or combinations thereof. In embodiments, the amount of the at least one humectant in the cleansing dye composition may be, based on a total weight of the cleansing dye composition, greater than or equal to 0.1 wt % and less than or equal to 3 wt %. In embodiments, the amount of the at least one humectant may be, based on a total weight of the cleansing dye composition, greater than or equal to 0.1 wt %, greater than or equal to 0.5 wt %, greater than or equal to 1 wt %, greater than or equal to 1.5 wt %, or even greater than or equal to 2 wt %. In embodiments, the amount of the at least one humectant in the cleansing dye composition may be, based on a total weight of the cleansing dye composition, less than or equal to 3 wt %, less than or equal to 2 wt %, less than or equal to 1 wt %, or even less than or equal to 0.5 wt %. In embodiments, the amount of the at least one humectant in the cleansing dye composition may be, based on a total weight of the cleansing dye composition, greater than or equal to 0.1 wt % and less than or equal to 3 wt %, greater than or equal to 0.1 wt % and less than or equal to 2 wt %, greater than or equal to 0.1 wt % and less than or equal to 1.5 wt %, greater than or equal to 0.1 wt % and less than or equal to 1 wt %, greater than or equal to 0.1 wt % and less than or equal to 0.5 wt %, greater than or equal to 0.5 wt % and less than or equal to 3 wt %, greater than or equal to 0.5 wt % and less than or equal to 2 wt %, greater than or equal to 0.5 wt % and less than or equal to 1.5 wt %, greater than or equal to 0.5 wt % and less than or equal to 1 wt %, greater than or equal to 1 wt % and less than or equal to 3 wt %, greater than or equal to 1 wt % and less than or equal to 2 wt %, greater than or equal to 1 wt % and less than or equal to 1.5 wt %, greater than or equal to 1.5 wt % and less than or equal to 3 wt %, greater than or equal to 1.5 wt % and less than or equal to 2 wt %, or even greater than or equal to 2 wt % and less than or equal to 3 wt %, or any and all sub-ranges formed from any of these endpoints.

Some embodiments of the compositions disclosed herein would readily lather with a quick flash, stable foam, and good cleansing ability-ranging from gentle cleansing to clarifying while maintaining high color deposition. For example, in embodiments, the cleansing dye composition may be formulated as a shampoo. In embodiments, the cleansing dye composition may be formulated as a body wash.

In embodiments, a method for coloring a keratin substrate may comprise applying the cleansing dye composition to the keratin substrate. For example, the cleansing dye composition may be applied to the keratin substrate using a user's hands or an applicator (e.g., brush), allowed to remain on the keratin substrate for a period of time (e.g., greater than or equal to 5 seconds and less than or equal to 20 minutes), and then rinsed off (e.g., with water).

### EXAMPLES

To enhance the understanding of various embodiments, reference is made to the following examples, which illustrate various embodiments of the cleansing dye compositions described herein.

Table 1 shows comparative example cleansing dye compositions C1-C3 and example cleansing dye compositions E1-E15 (in terms of wt %). It should be understood that “QS” indicates “quantum satis” and means that “just enough of the component to be sufficient” was added to the formulation. It should also be understood that “balance” means that enough of the component was added so as to bring the total of all components by weight to 100%.

TABLE 1

| | | | |
|---|---|---|---|
| Examples | C1 | C2 | C3 |

Cyclic saccharide-derived surfactant (wt % and total activity level (wt %))

| sodium laurylglucosides | — | — | — | |
|---|---|---|---|---|
| hydroxypropylsulfonate | | | | |
| (40% active) | | | | |
| cocoglucoside | — | — | — | |
| (50% active) | | | | |
| decyl glucoside | — | — | 12.0 | (6.0) |

(50% active)

Secondary surfactant (wt % and total activity level (wt %))

| sodium laureth sulfate | — | 12.9 | (9.0) | — | |
|---|---|---|---|---|---|
| (70% active) | | | | | |
| sodium lauryl sulfate | — | 13.3 | (4.0) | — | |
| (30% active) | | | | | |
| sodium lauroyl sarcosinate | — | — | | — | |
| (98% active) | | | | | |
| sodium cocoyl isethionate | — | — | | — | |
| (78% active) | | | | | |
| sodium lauryl sulfoacetate | — | — | | — | |
| (65% active) | | | | | |
| disodium laureth sulfosuccinate | — | — | | — | |
| (33% active) | | | | | |
| cocamidopropyl betaine | | 6.67 | (2.0) | 30.0 | (9.0) |

| | | | |
|---|---|---|---|
| (30% active) | | | |
| sodium C₁₄-C₁₆ olefin sulfonate | — | — | — |
| (40% active) | | | |
| sodium C₁₆ olefin sulfonate | — | — | — |
| (36% active) | | | |

Direct Dye (wt %)

| | | | |
|---|---|---|---|
| ext. D&C violet 2 | — | — | — |
| FD&C blue 1 | — | 0.1 | 0.1 |
| D&C red 33 | — | — | — |
| 2-amino-6-chloro-4-nitrophenol | — | — | — |
| HC blue 12 | — | — | — |
| basic violet 2 | — | — | — |

pH Adjuster (wt %)

| | | | |
|---|---|---|---|
| malic acid | — | — | — |
| citric acid | — | — | — |
| lactic acid | — | QS | QS |

Conditioning Agent (wt %)

| | | | |
|---|---|---|---|
| polyquaternium-7 | — | — | — |
| polyquaternium-39 | — | — | — |
| polyquaternium-10 | — | — | — |
| hydroxypropyl methylcellulose | — | — | — |
| guar hydroxypropyl-trimonium | — | — | — |
| chloride | | | |

Foaming Agent (wt %)

| | | | |
|---|---|---|---|
| PEG-12 dimethicone | — | — | — |
| polyester-11 | — | — | — |
| PPG-9 | — | — | — |
| water (and) silicone quaternium- | — | — | — |
| 18 (and) trideceth-6 (and) | | | |
| trideceth-12 | | | |
| cocamide MEA | — | — | — |
| cetyl alcohol | — | — | — |
| glycol distearate | — | — | — |

Humectant (wt %)

| | | | |
|---|---|---|---|
| glycerin | — | — | — |
| propanediol | — | — | — |
| butylene glycol | — | — | — |
| propylene glycol | — | — | — |
| pentylene glycol | — | — | — |
| benzyl alcohol | — | — | — |

Other (wt %)

| | | | |
|---|---|---|---|
| preservative | — | — | — |
| fragrance | — | — | — |
| water | — | balance | balance |

| | | | | | |
|---|---|---|---|---|---|
| Examples | E1 | E2 | E3 | E4 | E5 |

Cyclic saccharide-derived surfactant (wt % and total activity level (wt %))

| sodium laurylglucosides | — | | — | | — | 22.5 | (9.0) | — | |
|---|---|---|---|---|---|---|---|---|---|
| hydroxypropylsulfonate | | | | | | | | | |
| (40% active) | | | | | | | | | |
| cocoglucoside | 15.0 | (7.5) | 8.0 | (4.0) | — | 8.0 | (4.0) | 13.0 | (6.5) |

| (50% active) | | | | | | | | | |
|---|---|---|---|---|---|---|---|---|---|
| decyl glucoside | 15.0 | (7.5) | 18.0 | (9.0) | 18.0 | (9.0) | — | 13.0 | (6.5) |

(50% active)

Secondary surfactant (wt % and total activity level (wt %))

| sodium laureth sulfate | — | — | | — | | — | | — | |
|---|---|---|---|---|---|---|---|---|---|
| (70% active) | | | | | | | | | |
| sodium lauryl sulfate | — | — | | — | | — | | — | |
| (30% active) | | | | | | | | | |
| sodium lauroyl sarcosinate | — | — | | — | | — | | — | |
| (98% active) | | | | | | | | | |
| sodium cocoyl isethionate | — | — | | — | | — | | — | |
| (78% active) | | | | | | | | | |
| sodium lauryl sulfoacetate | — | — | | — | | — | | — | |
| (65% active) | | | | | | | | | |
| disodium laureth sulfosuccinate | — | — | | — | | — | | — | |
| (33% active) | | | | | | | | | |
| cocamidopropyl betaine | — | 6.67 | (2.0) | 13.3 | (4.0) | 6.67 | (2.0) | 6.67 | (2.0) |

| (30% active) | | | | | | |
|---|---|---|---|---|---|---|
| sodium C₁₄-C₁₆ olefin sulfonate | — | — | — | | — | — |
| (40% active) | | | | | | |
| sodium C₁₆ olefin sulfonate | — | — | 5.5 | (2.0) | — | — |

(36% active)

Direct Dye (wt %)

| | | | | | |
|---|---|---|---|---|---|
| ext. D&C violet 2 | — | — | — | — | — |
| FD&C blue 1 | 0.1 | 0.1 | 0.1 | 0.1 | 0.1 |
| D&C red 33 | — | — | — | — | — |
| 2-amino-6-chloro-4-nitrophenol | — | — | — | — | — |
| HC blue 12 | — | — | — | — | — |
| basic violet 2 | — | — | — | — | — |

pH Adjuster (wt %)

| | | | | | |
|---|---|---|---|---|---|
| malic acid | — | — | — | — | — |
| citric acid | — | — | — | — | — |
| lactic acid | QS | QS | QS | QS | QS |

Conditioning Agent (wt %)

| | | | | | |
|---|---|---|---|---|---|
| polyquaternium-7 | — | — | — | — | — |
| polyquaternium-39 | — | — | — | — | — |
| polyquaternium-10 | — | — | — | — | — |
| hydroxypropyl methylcellulose | — | — | — | — | — |
| guar hydroxypropyl-trimonium | — | — | — | — | — |
| chloride | | | | | |

Foaming Agent (wt %)

| | | | | | |
|---|---|---|---|---|---|
| PEG-12 dimethicone | — | — | — | — | — |
| polyester-11 | — | — | — | — | — |
| PPG-9 | — | — | — | — | — |
| water (and) silicone quaternium- | — | — | — | — | — |
| 18 (and) trideceth-6 (and) | | | | | |
| trideceth-12 | | | | | |
| cocamide MEA | — | — | — | — | — |
| cetyl alcohol | — | — | — | — | — |
| glycol distearate | — | — | — | — | — |

Humectant (wt %)

| | | | | | |
|---|---|---|---|---|---|
| glycerin | — | — | — | — | — |
| propanediol | — | — | — | — | — |
| butylene glycol | — | — | — | — | — |
| propylene glycol | — | — | — | — | — |
| pentylene glycol | — | — | — | — | — |
| benzyl alcohol | — | — | — | — | — |

Other (wt %)

| preservative | — | — | — | — | — |
|---|---|---|---|---|---|
| fragrance | — | — | — | — | — |
| water | balance | balance | balance | balance | balance |
| Examples | E6 | E7 | E8 | E9 | E10 |

Cyclic saccharide-derived surfactant (wt % and total activity level (wt %))

| sodium laurylglucosides | — | | — | 22.5 | (9.0) | — | | — | |
|---|---|---|---|---|---|---|---|---|---|
| hydroxypropylsulfonate | | | | | | | | | |
| (40% active) | | | | | | | | | |
| cocoglucoside | 15.0 | (7.5) | — | 8.0 | (4.0) | 8.0 | (4.0) | 13.0 | (6.5) |

| (50% active) | | | | | | | | | |
|---|---|---|---|---|---|---|---|---|---|
| decyl glucoside | 15.0 | (7.5) | 18.0 | (9.0) | — | 18.0 | (9.0) | 13.0 | (6.5) |

(50% active)

Secondary surfactant (wt % and total activity level (wt %))

| sodium laureth sulfate | — | — | | — | | — | | — | |
|---|---|---|---|---|---|---|---|---|---|
| (70% active) | | | | | | | | | |
| sodium lauryl sulfate | — | — | | — | | — | | — | |
| (30% active) | | | | | | | | | |
| sodium lauroyl sarcosinate | — | — | | — | | — | | — | |
| (98% active) | | | | | | | | | |
| sodium cocoyl isethionate | — | — | | — | | — | | — | |
| (78% active) | | | | | | | | | |
| sodium lauryl sulfoacetate | — | — | | — | | — | | — | |
| (65% active) | | | | | | | | | |
| disodium laureth sulfosuccinate | — | — | | — | | — | | — | |
| (33% active) | | | | | | | | | |
| cocamidopropyl betaine | — | 13.3 | (4.0) | 6.67 | (2.0) | 6.67 | (2.0) | 6.67 | (2.0) |

| (30% active) | | | | | | |
|---|---|---|---|---|---|---|
| sodium C₁₄-C₁₆ olefin sulfonate | — | — | | — | — | — |
| (40% active) | | | | | | |
| sodium C₁₆ olefin sulfonate | — | 5.5 | (2.0) | — | — | — |

(36% active)

Dye (wt %)

| | | | | | |
|---|---|---|---|---|---|
| ext. D&C violet 2 | — | 0.05 | — | 0.05 | — |
| FD&C blue 1 | — | 0.05 | — | — | 0.1 |
| D&C red 33 | — | — | — | 0.05 | — |
| 2-amino-6-chloro-4-nitrophenol | — | — | 0.1 | — | — |
| HC blue 12 | — | — | 0.01 | — | — |
| basic violet 2 | 0.001 | — | 0.001 | — | — |

pH Adjuster (wt %)

| | | | | | |
|---|---|---|---|---|---|
| malic acid | — | — | — | — | — |
| citric acid | 0.5 | 0.5 | 0.5 | 0.5 | 1.0 |
| lactic acid | 0.15 | 0.15 | 0.15 | 0.15 | 0.5 |

Conditioning Agent (wt %)

| | | | | | |
|---|---|---|---|---|---|
| polyquaternium-7 | 2.0 | 1.5 | 1.5 | 1.5 | 2.0 |
| polyquaternium-39 | — | — | — | — | — |
| polyquaternium-10 | 0.25 | 0.20 | 0.20 | 0.20 | — |
| hydroxypropyl methylcellulose | 0.20 | 0.20 | 0.20 | 0.20 | — |
| guar hydroxypropyl-trimonium | 0.10 | 0.20 | 0.20 | 0.20 | — |
| chloride | | | | | |

Foaming Agent (wt %)

| | | | | | |
|---|---|---|---|---|---|
| PEG-12 dimethicone | — | — | — | — | 0.3 |
| polyester-11 | — | — | — | — | 1.0 |
| PPG-9 | 0.01 | 0.01 | 0.01 | 0.01 | 0.01 |
| water (and) silicone quaternium- | — | — | — | — | — |
| 18 (and) trideceth-6 (and) | | | | | |
| trideceth-12 | | | | | |
| cocamide MEA | 0.5 | 1.0 | 1.0 | 1.0 | 0 |
| cetyl alcohol | — | — | — | — | 0.3 |
| glycol distearate | 1.5 | 2.0 | 2.0 | 2.0 | 1.0 |

Humectant (wt %)

| | | | | | |
|---|---|---|---|---|---|
| glycerin | | 1.0 | — | 1.0 | 0.5 |
| propanediol | 0.8 | — | — | — | 0.75 |
| butylene glycol | 0.8 | — | — | — | 0.5 |
| propylene glycol | — | — | 0.5 | — | — |
| pentylene glycol | — | 1.0 | — | 1.0 | — |
| benzyl alcohol | — | — | — | — | 0.25 |

Other (wt %)

| | | | | | |
|---|---|---|---|---|---|
| preservative | QS | QS | QS | QS | QS |
| fragrance | QS | QS | QS | QS | QS |

| Examples | E11 | | E12 | E13 | E14 | E15 |
|---|---|---|---|---|---|---|
| Cyclic saccharide-derived surfactant (wt % and total activity level (wt %)) | | | | | | |
| sodium laurylglucosides | 10 | (4.0) | — | — | — | — |

hydroxypropylsulfonate
(40% active)

| | | | | | | | | |
|---|---|---|---|---|---|---|---|---|
| cocoglucoside | — | 6.0 | (3.0) | — | 9.0 | (4.5) | 9.0 | (4.5) |

(50% active)

| | | | | | | | | | | |
|---|---|---|---|---|---|---|---|---|---|---|
| decyl glucoside | 12.0 | (6.0) | 20 | (10) | 18.0 | (9.0) | 8.0 | (4.0) | 9.0 | (4.5) |

| (50% active) | | | | | |
|---|---|---|---|---|---|
| Secondary surfactant (wt % and total activity level (wt %)) | | | | | |
| sodium laureth sulfate | — | — | — | — | — |
| (70% active) | | | | | |
| sodium lauryl sulfate | — | — | — | — | — |
| (30% active) | | | | | |

| | | | | | | | | | |
|---|---|---|---|---|---|---|---|---|---|
| sodium lauroyl sarcosinate | 2.04 | (2.0) | — | 4.08 | (4.0) | 2.04 | (2.0) | 2.04 | (2.0) |

(98% active)

| | | | | | | | |
|---|---|---|---|---|---|---|---|
| sodium cocoyl isethionate | — | — | — | 2.56 | (2.0) | 2.56 | (2.0) |

| (78% active) | | | | | | |
|---|---|---|---|---|---|---|
| sodium lauryl sulfoacetate | 3.08 | (2.0) | — | — | — | — |
| (65% active) | | | | | | |
| disodium laureth sulfosuccinate | 3.0 | (1.0) | — | — | — | — |

(33% active)

| | | | | | | | | |
|---|---|---|---|---|---|---|---|---|
| cocamidopropyl betaine | — | — | 6.67 | (2.0) | 15.0 | (7.5) | 6.67 | (2.0) |

| (30% active) | | | | | | |
|---|---|---|---|---|---|---|
| sodium C₁₄-C₁₆ olefin sulfonate | — | — | — | 5.0 | (2.0) | — |

| (40% active) | | | | | |
|---|---|---|---|---|---|
| sodium C₁₆ olefin sulfonate | — | — | — | — | — |
| (36% active) | | | | | |
| Dye (wt %) | | | | | |
| ext. D&C violet 2 | 0.1 | — | — | 0.05 | 0.05 |
| FD&C blue 1 | — | — | — | 0.05 | 0.001 |
| D&C red 33 | — | — | — | — | 0.006 |
| 2-amino-6-chloro-4-nitrophenol | — | 0.3 | — | — | — |
| HC blue 12 | — | 0.01 | 0.1 | — | — |
| basic violet 2 | — | — | 0.01 | — | — |
| pH Adjuster (wt %) | | | | | |
| malic acid | 1.0 | 1.0 | — | — | — |
| citric acid | — | — | 0.5 | 0.5 | 0.5 |
| lactic acid | 0.5 | 0.5 | 0.15 | 0.15 | 0.15 |
| Conditioning Agent (wt %) | | | | | |
| polyquaternium-7 | — | — | 1.5 | 1.5 | 1.5 |
| polyquaternium-39 | — | — | — | — | — |
| polyquaternium-10 | 0.20 | 0.30 | 0.20 | 0.20 | 0.20 |
| hydroxypropyl methylcellulose | — | — | 0.20 | 0.20 | 0.20 |
| guar hydroxypropyl-trimonium | 0.20 | — | 0.20 | 0.20 | 0.20 |
| chloride | | | | | |
| Foaming Agent (wt %) | | | | | |
| PEG-12 dimethicone | 0.05 | — | — | — | — |
| polyester-11 | — | — | — | — | — |
| PPG-9 | 0.01 | 0.01 | 0.01 | 0.01 | 0.01 |
| water (and) silicone quaternium- | — | 0.50 | — | — | — |
| 18 (and) trideceth-6 (and) | | | | | |
| trideceth-12 | | | | | |
| cocamide MEA | 1.0 | 1.0 | 1.0 | 1.0 | 1.0 |
| cetyl alcohol | 1.5 | 0.15 | — | — | — |
| glycol distearate | 2.0 | 0.35 | 2.0 | 2.0 | 2.0 |
| Humectant (wt %) | | | | | |
| glycerin | 0.5 | — | 0.3 | 0.3 | — |
| propanediol | 0.5 | 0.8 | — | — | — |
| butylene glycol | 0.75 | 0.5 | — | — | — |
| propylene glycol | — | — | — | — | — |
| pentylene glycol | — | — | — | — | 1.0 |
| benzyl alcohol | 0.25 | — | — | — | — |
| Other (wt %) | | | | | |
| preservative | QS | QS | QS | QS | QS |
| fragrance | QS | QS | QS | QS | QS |
| water | balance | balance | balance | balance | balance |

Referring now to Table 2, comparative example blonde goat tress CT1 was an untreated 1 g blonde goat tress. 1 g comparative example blonde goat tresses CT2-CT3 and example blonde goat tresses ET1-ET5 were placed in a comparative example or an example cleansing dye composition, as indicated in Table 2, in a container at room temperature for 5 minutes, rinsed with deionized water, and allowed to dry. As shown in Table 1, each cleansing dye composition included 0.1 wt % Blue 1 direct dye. The pH of each solution was adjusted to about 3.75. The measured ΔE values and b* values of the comparative example and example blonde goat tresses are also shown in Table 2.

TABLE 2

| Examples | CT1 | CT2 | CT3 | ET1 | ET2 | ET3 | ET4 | ET5 |
|---|---|---|---|---|---|---|---|---|
| Composition | C1 | C2 | C3 | E1 | E2 | E3 | E4 | E5 |
| ΔE value | — | 1.5 | 1.9 | 11.1 | 8.4 | 5.8 | 3.7 | 14.5 |
| b* value | 13.08 | 13.06 | 12.59 | 4.23 | 8.14 | 9.35 | 11.27 | 6.34 |

As shown in Table 2, example tresses ET1-ET5, tresses treated with cleansing dye compositions including 30 wt %, 26 wt %, 18 wt %, 30.5 wt %, and 26 wt %, respectively, of at least one of sodium laurylglucosides hydroxypropylsulfonate, cocoglucoside, and decyl glucoside, had greater ΔE values and lower b* values than comparative example tresses CT2 and CT3, tresses treated with cleansing dye compositions including 0 wt % cyclic saccharide-derived surfactant and 12.0 wt % decyl glucoside, respectively. As exemplified by comparative example tresses CT1-CT3 and example tresses ET1-ET5, cleansing dye compositions including greater than 15 wt % of at least one cyclic saccharide-derived surfactant have improved dye deposition.

Embodiments may be further described with respect to the below clauses:
1. A cleansing dye composition comprising, based on a total weight of the cleansing dye composition, greater than or equal to 0.0001 wt % and less than or equal to 10 wt % of at least one direct dye; and greater than 15 wt % and less than or equal to 50 wt % of at least one cyclic saccharide-derived surfactant having a total surfactant activity level greater than 7.5 wt % and less than or equal to 25 wt %.
2. The cleansing dye composition of clause 1, wherein the cleansing dye composition comprises, based on the total weight of the cleansing dye composition, greater than or equal to 17 wt % and less than or equal to 45 wt %, greater than or equal to 19 wt % and less than or equal to 40 wt %, or even greater than or equal to 21 wt % and less than or equal to 35 wt % of the at least one cyclic saccharide-derived surfactant.
3. The cleansing dye composition of any preceding clause, wherein the total surfactant activity level of the at least one cyclic saccharide-derived surfactant is, based on the total weight of the cleansing dye composition, greater than or equal to 8.5 wt % and less than or equal to 21 wt %, greater than or equal to 9.5 wt % and less than or equal to 19 wt %, or even greater than or equal to 10.5 wt % and less than or equal to 17 wt %.
4. The cleansing dye composition of any preceding clause, wherein the at least one cyclic saccharide-derived surfactant comprises decyl glucoside, coco glucoside, lauryl glucoside, sodium laurylglucoside hydroxypropylsulfonate, or combinations thereof.
5. The cleansing dye composition of any preceding clause, wherein the cleansing dye composition further comprises at least one secondary surfactant.
6. The cleansing dye composition of any preceding clause, wherein the secondary surfactant comprises betaines, imidazoline derivatives, sarcosinates, sulfosuccinates, sulfates, sulfonates, amino acid-derived surfactants, isethionates, polysorbates, carboxylates, dimeric surfactants, alkoxylates, silicon surfactants, cationic surfactants, or combinations thereof.
7. The cleansing dye composition of any preceding clause, wherein the secondary surfactant comprises alkyl betaines, alkyl amido alkyl betaines, sulfo betaines, alkyl sulfates, alkyl ether sulfates, alkyl sulfonates, glutamates, fatty alcohol ethoxylates, alkyl phenol ethoxylates, fatty acid alkoxylates, alkyl ether carboxylates, silicon polyethers, quaternary ammonium compounds, amine salts, or combinations thereof.
8. The cleansing dye composition of any preceding clause, wherein the secondary surfactant comprises cocamidopropyl betaine, sodium lauroyl sarcosinate, sodium cocoyl isethionate, sodium lauryl sulfoacetate, disodium laureth sulfosuccinate, sodium C₁₄-C₁₆ olefin sulfonate, sodium C₁₆ olefin sulfonate, or combinations thereof.
9. The cleansing dye composition of any preceding clause, wherein the cleansing dye composition comprises a greater amount of the at least one cyclic saccharide-derived surfactant than the at least one secondary surfactant, based on the total weight of the cleansing dye composition.
10. The cleansing dye composition of any preceding clause, wherein the cleansing dye composition comprises a weight ratio of the at least one cyclic saccharide-derived surfactant to the at least one secondary surfactant of 1:1 to 5:1.
11. The cleansing dye composition of any preceding clause, wherein the cleansing dye composition comprises, based on the total weight of the cleansing dye composition, greater than or equal to 1 wt % and less than or equal to 25 wt % of the secondary surfactant.
12. The cleansing dye composition of any preceding clause, wherein a total amount of the at least one cyclic saccharide-derived surfactant and the at least one secondary surfactant is, based on the total weight of the cleansing dye composition, greater than or equal to 16 wt % and less than or equal to 50 wt %.
13. The cleansing dye composition of any preceding clause, wherein a total surfactant activity level of the at least one cyclic saccharide-derived surfactant and the at least one secondary surfactant is, based on the total weight of the cleansing dye composition, greater than or equal to 8 wt % and less than or equal to 25 wt %.
14. The cleansing dye composition of any preceding clause, wherein the at least one direct dye comprises an acidic dye.
15. The cleansing dye composition of any preceding clause, wherein the at least one direct dye comprises Ext. Violet 2/Acid Violet 43, Blue 1/Acid Blue 9, FD&C Green 3, D&C Green 5, D&C Green 6, D&C Green 8, D&C Yellow 8, Yellow 11, Yellow 10/Acid Yellow 3, Yellow 5/Acid Yellow 23, FD&C Yellow 6/E110, Orange 4/Acid Orange 7, Orange 5, Red 6 FD&C Red 40, D&C Red 4, D&C Red 6, D&C Red 17, D&C Red 33/Acid Red 33, D&C Red 27, D&C Red 28, Violet 2, HC Blue 2, 2-amino-6-chloro-4-nitrophenol, HC Red No. 3, HC Blue No. 12, 4-hydroxypropylamino-3-nitrophenol, Basic Yellow 57, Basic Red 76, Basic Blue 124, Basic Violet 2, Basic Blue 99, HC Blue 16, Basic Orange 31, Basic Brown 17, Basic Red 51, Basic Yellow 87 or combinations thereof.
16. The cleansing dye composition of any preceding clause, wherein the cleansing dye composition comprises, based on the total weight of the cleansing dye composition, greater than or equal to 0.001 wt % and less than or equal to 7 wt %, greater than or equal to 0.005 wt % and less than or equal to 5 wt %, greater than or equal to 0.01 wt % and less than or equal to 3 wt %, or greater than or equal to 0.05 wt % and less than or equal to 1 wt % of the at least one direct dye.
17. The cleansing dye composition of any preceding clause, wherein the cleansing dye composition further comprises, based on the total weight of the cleansing dye composition, greater than or equal to 0.05 wt % and less than or equal to 2 wt % of at least one pH adjuster.
18. The cleansing dye composition of any preceding clause, wherein the cleansing dye composition further comprises, based on the total weight of the cleansing dye composition, greater than or equal to 0.05 wt % and less than or equal to 5 wt % of at least one conditioning agent.
19. The cleansing dye composition of any preceding clause, wherein the cleansing dye composition further comprises, based on the total weight of the cleansing dye composition, greater than or equal to 0.005 wt % and less than or equal to 5 wt % of at least one foaming agent.
20. The cleansing dye composition of any preceding clause, wherein the cleansing dye composition further comprises, based on the total weight of the cleansing dye composition, greater than or equal to 0.1 wt % and less than or equal to 3 wt % of at least one humectant.
21. The cleansing dye composition of any preceding clause, wherein the cleansing dye composition is formulated for use as a shampoo.
22. A method for coloring a keratin substrate, comprising applying to the keratin substrate the cleansing dye composition of any preceding clause.

What is claimed is:

1. A cleansing dye composition comprising, based on a total weight of the cleansing dye composition:
greater than or equal to 0.0001 wt % and less than or equal to 10 wt % of at least one direct dye; and
greater than 15 wt % and less than or equal to 50 wt % of at least one cyclic saccharide-derived surfactant having a total surfactant activity level greater than 7.5 wt % and less than or equal to 25 wt %.

2. The cleansing dye composition of claim 1, wherein the cleansing dye composition comprises, based on the total weight of the cleansing dye composition, greater than or equal to 17 wt % and less than or equal to 45 wt %, greater than or equal to 19 wt % and less than or equal to 40 wt %, or even greater than or equal to 21 wt % and less than or equal to 35 wt % of the at least one cyclic saccharide-derived surfactant.

3. The cleansing dye composition of claim 1, wherein the total surfactant activity level of the at least one cyclic saccharide-derived surfactant is, based on the total weight of the cleansing dye composition, greater than or equal to 8.5 wt % and less than or equal to 21 wt %, greater than or equal to 9.5 wt % and less than or equal to 19 wt %, or even greater than or equal to 10.5 wt % and less than or equal to 17 wt %.

4. The cleansing dye composition of claim 1, wherein the at least one cyclic saccharide-derived surfactant comprises decyl glucoside, coco glucoside, lauryl glucoside, sodium lauryl glucoside hydroxypropylsulfonate, or combinations thereof.

5. The cleansing dye composition of claim 1, wherein the cleansing dye composition further comprises at least one secondary surfactant.

6. The cleansing dye composition of claim 5, wherein the secondary surfactant comprises betaines, imidazoline derivatives, sarcosinates, sulfosuccinates, sulfates, sulfonates, amino acid-derived surfactants, isethionates, polysorbates, carboxylates, dimeric surfactants, alkoxylates, silicon surfactants, cationic surfactants, or combinations thereof.

7. The cleansing dye composition of claim 5, wherein the secondary surfactant, wherein the secondary surfactant comprises alkyl betaines, alkyl amido alkyl betaines, sulfo betaines, alkyl sulfates, alkyl ether sulfates, alkyl sulfonates, glutamates, fatty alcohol ethoxylates, alkyl phenol ethoxylates, fatty acid alkoxylates, alkyl ether carboxylates, silicon polyethers, quaternary ammonium compounds, amine salts, or combinations thereof.

8. The cleansing dye composition of claim 5, wherein the secondary surfactant comprises cocamidopropyl betaine, sodium lauroyl sarcosinate, sodium cocoyl isethionate, sodium lauryl sulfoacetate, disodium laureth sulfosuccinate, sodium C₁₄-C₁₆ olefin sulfonate, sodium C₁₆ olefin sulfonate, or combinations thereof.

9. The cleansing dye composition of claim 5, wherein the cleansing dye composition comprises a greater amount of the at least one cyclic saccharide-derived surfactant than the at least one secondary surfactant, based on the total weight of the cleansing dye composition.

10. The cleansing dye composition of claim 5, wherein the cleansing dye composition comprises a weight ratio of the at least one cyclic saccharide-derived surfactant to the at least one secondary surfactant of 1:1 to 5:1.

11. The cleansing dye composition of claim 5, wherein the cleansing dye composition comprises, based on the total weight of the cleansing dye composition, greater than or equal to 1 wt % and less than or equal to 25 wt % of the secondary surfactant.

12. The cleansing dye composition of claim 5, wherein a total amount of the at least one cyclic saccharide-derived surfactant and the at least one secondary surfactant is, based on the total weight of the cleansing dye composition, greater than or equal to 16 wt % and less than or equal to 50 wt %.

13. The cleansing dye composition of claim 5, wherein a total surfactant activity level of the at least one cyclic saccharide-derived surfactant and the at least one secondary surfactant is, based on the total weight of the cleansing dye composition, greater than or equal to 8 wt % and less than or equal to 25 wt %.

14. The cleansing dye composition of claim 1, wherein the at least one direct dye comprises an acidic dye.

15. The cleansing dye composition of claim 1, wherein the at least one direct dye comprises Ext. Violet 2/Acid Violet 43, Blue 1/Acid Blue 9, FD&C Green 3, D&C Green 5, D&C Green 6, D&C Green 8, D&C Yellow 8, Yellow 11, Yellow 10/Acid Yellow 3, Yellow 5/Acid Yellow 23, FD&C Yellow 6/E110, Orange 4/Acid Orange 7, Orange 5, Red 6 FD&C Red 40, D&C Red 4, D&C Red 6, D&C Red 17, D&C Red 33/Acid Red 33, D&C Red 27, D&C Red 28, Violet 2, HC Blue 2, 2-amino-6-chloro-4-nitrophenol, HC Red No. 3, HC Blue No. 12, 4-hydroxypropylamino-3-nitrophenol, Basic Yellow 57, Basic Red 76, Basic Blue 124, Basic Violet 2, Basic Blue 99, HC Blue 16, Basic Orange 31, Basic Brown 17, Basic Red 51, Basic Yellow 87 or combinations thereof.

16. The cleansing composition of claim 1, wherein the cleansing dye composition comprises, based on the total weight of the cleansing dye composition, greater than or equal to 0.001 wt % and less than or equal to 7 wt %, greater than or equal to 0.005 wt % and less than or equal to 5 wt %, greater than or equal to 0.01 wt % and less than or equal to 3 wt %, or greater than or equal to 0.05 wt % and less than or equal to 1 wt %, of the at least one direct dye.

17. The cleansing dye composition of claim 1, wherein the cleansing dye composition further comprises, based on the total weight of the cleansing dye composition, greater than or equal to 0.05 wt % and less than or equal to 2 wt % of at least one pH adjuster.

18. The cleansing dye composition of claim 1, wherein the cleansing dye composition further comprises, based on the total weight of the cleansing dye composition, greater than or equal to 0.05 wt % and less than or equal to 5 wt % of at least one conditioning agent.

19. The cleansing dye composition of claim 1, wherein the cleansing dye composition further comprises, based on the total weight of the cleansing dye composition, greater than or equal to 0.005 wt % and less than or equal to 5 wt % of at least one foaming agent.

20. The cleansing dye composition of claim 1, wherein the cleansing dye composition further comprises, based on the total weight of the cleansing dye composition, greater than or equal to 0.1 wt % and less than or equal to 3 wt % of at least one humectant.
